(19) United States
(12) Patent Application Publication
(10) Pub. No.: US 2004/0131771 A1
(43) Pub. Date: Jul. 8, 2004

(54) Coating of particulate material with organic based coating composition for the preparation of drug delivery systems

(76) Inventors: Poul Egon Bertelsen, Vanlose (DK); Karin Lowenstein Christensen, Frederiksberg (DK); Pernille Nybo, Rodovre (DK); Annmari Djuurhus, Ringsted (DK); Jimmy Hirschsprung Schlyter, Greve (DK)

Correspondence Address: EDWARDS & ANGELL, LLP, P.O. BOX 55874, BOSTON, MA, 02205, US

(21) Appl. No.: 10/701,720

(22) Filed: Nov. 4, 2003

Related U.S. Application Data
(60) Provisional application No. 60/423,785, filed on Nov. 4, 2002.

(51) Int. Cl. A61L 2/00; B05D 1/02; B05D 7/00

(52) U.S. Cl. 427/212; 427/421; 427/2.1

(57) ABSTRACT

A method for coating a particulate material for pharmaceutical, cosmeceutical, nutriceutical or cosmetic use or for use in food or food stuff, the coating being performed in a coating equipment, which comprises a coating chamber having

i) means for supply of a coating composition, and

ii) means for supply of inlet air to provide a flow of inlet air,

the method comprises

i) loading uncoated or pre-coated particulate material into the coating chamber,

ii) providing a flow of inlet air that has been adjusted so that the humidity of the air in the coating chamber ensures that unwanted agglomeration of the particulate material and/or adherence to the coating equipment are substantially reduced or avoided during the coating process, and

iii) spraying on the particulate material a coating composition comprising a solvent that contains at least about 70% v/v of one or more organic solvents and at the most about 30% v/v of an aqueous medium,

to obtain coated particulate material containing at the most about 20% w/w agglomerates.

The method is especially suitable in order to enable coating by means of an organic solvent based coating composition and avoids generally observed problems with respect to static electricity, adherence and formation of agglomerates.

### FIELD OF THE INVENTION

The present invention relates to a method for coating particulate material for pharmaceutical, cosmeceutical, nutriceutical, or cosmetic use or for use in the preparation of food or foodstuff. The method is especially suitable for use in those situations where an aqueous based coating composition is not appropriate, but where an organic solvent coating composition is applicable. Such a situation may occur when preparing a specific drug delivery system comprising a substance, which is sensitive towards water and/or aqueous media, which means that the exposure to water and/or aqueous media should be controlled.

Furthermore, an organic based coating is often compared with aqueous based coatings as it is possible to obtain a coating that has improved properties with respect to strength and retardation and, moreover, it gives better possibilities of adding hydrophobic excipients like e.g. paraffin, cutina etc. Another advantage is that it is possible to incorporate e.g. poorly water-soluble/not water-soluble active substance in the coating composition.

The method of the present invention is especially designed to avoid problems with respect to static electricity that leads to adherence of the particulate material to the coating equipment and/or other particles. Furthermore, such problems may lead to poor reproducibility, poor yield and/or insufficient and/or uneven coating. The method of the present invention also takes into account that the particulate material must not be overwetted. Both situations (i.e. static electricity and overwetting) might lead to unwanted agglomeration of the particulate material.

The method of the present invention provides a specific range with respect to the relative humidity of the air in the coating chamber during the coating process.

### BACKGROUND OF THE INVENTION

In the recent years, there has been focus on developing coating processes utilizing aqueous based coating composition. However, in certain cases it is not appropriate to use an aqueous based coating composition due to certain limitations.

Many therapeutically, prophylactically and/or diagnostically active substances are poorly water-soluble or not water-soluble and/or sensitive to water. Thus, an increasing number of new active substances (NCEs) are lipophilic or have a low solubility in water. Furthermore, a number of pharmaceutically acceptable excipients, e.g. certain cellulose derivatives, are also sensitive to water and e.g. swell upon contact with water. When such substances are used in a drug delivery system such as e.g. a solid dosage form then it is often desired to apply a coating that does not contain water or only a small amount of water. Especially, In those situations where it is desired to include an amount of a poorly water-soluble, water-insoluble and/or water-sensitive substance (e.g. an active substance) in a coating, a water-based coating composition may be a disadvantage e.g. from a solubility or stability point of view. Thus, a manufacturing process employing no aqueous medium or only a small amount of aqueous medium should be advantageous. In other cases, a coating is desired that has properties different from those that can be obtained by use of a water-based coating composition. In general, it is believed that a stronger or more firm coating can be obtained by use of organic solvent based coating, i.e. it is possible to obtain a coating that enables an increase or delay in release of the active substance contained in the solid dosage form. Furthermore, an advantage of using an organic based coating composition compared to that of a water based coating composition is that a lesser amount of film-forming polymer is needed in order to obtain a suitable film.

However, the present inventors have found that the use of organic solvent based coating compositions creates another problem, namely problems with respect to static electricity that may lead to unwanted agglomeration of the particulate material.

### DESCRIPTION OF THE INVENTION

Accordingly, the present invention relates to a method for coating a particulate material for pharmaceutical or cosmetic use or for use in the preparation of food. The coating is performed in coating equipment, which comprises a coating chamber having

i) means for supply of a coating composition, and

ii) means for supply of inlet air to provide a flow of inlet air,

the method comprises

i) loading uncoated or precoated particulate material into the coating chamber,

ii) providing a flow of inlet air that has been adjusted so that the humidity of the air in the coating chamber ensures that unwanted agglomeration of the particulate material and/or adherence to the coating equipment are substantially reduced or avoided during the coating process, and

iii) spraying on the particulate material a coating composition comprising a solvent that, contains at least about 70% v/v of one or more organic solvents and at the most about 30% v/v of an aqueous medium,

to obtain coated particulate material containing at the most about 20% w/w agglomerates (determined as described In the paragraph headed “Methods”).

In general the coating process can be performed in any suitable coating equipment such as, e.g., a fluid bed (e.g. top spray, bottom spray, tangential spray), a spray dryer (e.g. co current, counter current) or a side-vented coating pan.

In general, coating with an organic solvent based composition leads to a stronger film than when a water based coating composition is used. By using the present method it is possible to avoid undesired agglomeration of particles during the coating process, which is a clear advantage form a process economical point of view. Furthermore, the process is applicable for all kinds of active substance irrespective e.g. of their solubility and the coating process ensures an even distribution of the film on the particulate material.

In a method according to the invention, the mean particle size of the uncoated particulate material is at the most about 1400 μm. In particular embodiments, the particle size of the uncoated particulate material is at the most about 1200 μm such as, e.g., at the most about 1100 μm, at the most about 1000 μm, at the most about 900 μm, at the most about 800 μm, at the most about 750 am, at the most about 700 μm, at the most about 650 μm, at the most about 600 μm, at the most about 550 μm or at the most about 500 μm; such as, e.g., from about 150 μm to about 1200 μm, from about 200 μm to about 1200 μm from about 200 μm to about 1000 μm, from about 250 μm to about 800 μm or from about 300 μm to about 750 μm. In a specific embodiment of the Invention the particle size of the core is at the most about 500 μm to about 1000 μm, or from about 350 μm to about 500 μm.

In some cases, the particle size of the particulate material may be even higher. Thus, in some cases the particle size may be at the most about 2 mm.

The shape of the particles may be any suitable shape including a rounded or oval shape as well as a polygonal or rod-like or flake-like shape.

The density of the particulate material including pores is generally below about 3 g/cm³ such as, e.g., below about 2.8 g/cm³, below about 2.5 g/cm³, below about 2.3 g/cm³, below about 2.0 g/cm³, below about 1.8 g/cm³, below about 1.75 g/cm³, below about 1.6 g/cm⁵, at the most about 1.55 g/cm³, at the most about 1.5 g/cm³ or at the most about 1-4 g/cm³. The density of the particulate material is determined by standard methods known In the art such as, e.g., mercury intrusion. In the case of sucrose or cellulose based beads, the density is generally about 1.5 g/cm³. However, the two different types of beads behave very different in a coating process, which may be related to the difference in physico-chemical properties (hydrophilicity, water content etc.). Accordingly, the coating conditions may be adjusted to each specific particular material employed. In the examples herein specific conditions are given that lead to suitable results.

A method according to the Invention may also be applied for coating of particulate material that has a relatively low density such as, e.g., a density of about 1-10 mg/cm³.

In a specific embodiment, the particulate material that is coated by the method according to the invention is typically a material that has a mean particle size of at the most about 1000 μm and a density of at the most about 2 g/cm³ (density for particulate material including pores).

When coating particulate material like e.g. pellets it is the objective to supply each pellet with a uniform layer of film giving a well-defined and controllable release of the active substance. Therefore, agglomeration of pellets or adherence of pellets to the walls of the coating equipment may make it impossible to meet predetermined requirements such as, e.g., dissolution requirements, yields, particle size etc.

An example of such a material is cellulose spheres, which revealed many problems when subject to organic based coating. In general agglomeration or adherence will occur if the coating process is carried out under too wet conditions with a too high liquid flow rate. However, when dealing with an organic based coating process, agglomeration and/or adherence can also occur caused by static electricity. Static electricity will occur if the relative humidity in the coating chamber is too low. Reducing the problem with the static electricity can sometimes be solved by e.g. Increasing the process air flow with the purpose of making the coating process more vigorous and/or by making the process more wet by increasing the liquid flow rate. However, neither of these suggestions was optimal in the present case.

Sometimes, problems relating to static electricity can be overcome by using different approaches such as, e.g. addition of an increased amount of talc or other similar agents, reduction of the polymer content in the coating composition, use of a polymer with a lower standard viscosity in the coating composition and/or use different solvent systems having a higher evaporation rate (e.g. acetone). However, addition of one or more excipients to the coating composition may influence the properties of the resulting coating. Thus, addition of e.g. macrogol (also denoted polyethylene glycol or PEG) leads to formation of pores upon contact with water and influences the degree of retardation. All these different approaches have been investigated and the only one that seems to solve some of the problems is to lower the polymer concentration. However, it is not optimal as it generally leads to too long coating times (more than about 7 hours) and more solvent is needed, which leads to an uneconomical process and waste problems.

Accordingly, other alternative solutions are necessary in order to solve the problem with adherence and/or unwanted agglomeration. The inventors have found that having the inlet air carrying an amount of water in a certain interval leading to a specific range of relative humidity in the coating chamber will lead to the desired result by which the static electricity of the particulate material has a level that does not lead to agglomeration of the particulate material by overwetting and/or increased stickiness of the polymer/film.

In other words, a solution to the problem is achieved by increasing the relative humidity, RH, in the coating chamber by adjusting the amount of water in the inlet air or the coating liquid. However, the level of RH in the coating chamber is very critical and the right level cannot be predicted easily. Too low RH will not eliminate the static electricity and too high RH may damage the film formation and may furthermore lead to agglomeration of the pellets. The latter situation is seen for e.g. film-forming polymers having a medium/high standard viscosity (standard viscosity for ethylcellulose is determined for a 5% solution at 25° C. in 80% toluene and 20% ethanol), Furthermore, a suitable relative humidity may depend on the specific type of film-forming polymer employed as well as the coating equipment, the type and size of particulate material, addition of one or more excipients like e.g. anti-adhesive excipients etc. From the description, methods and examples herein, a person skilled in the art has guidance of how to adjust the relative humidity. In order to achieve a suitable result taken the above-mentioned variable conditions into account

Furthermore, the present inventors have developed a method that makes it possible easily to determine the lower RH limit. This method involves the use of a coloring agent that is applied in the form of a coating composition during the coating process. Particles that adhere to the coating equipment due to e.g. static electricity are not moved continuously during the coating process and will not be coated and, accordingly, not be colored or not fully colored. Normally, this test is passed for a specific particulate material if at the most 5-10% of the material is not colored or not fully colored during the normal coating conditions. The method is described in more details herein in the paragraph headed “Methods”.

As mentioned above, the humidity of the air in the coating chamber in a method of the invention must be adjusted to a range that results in coated particulate material, wherein the percentage of oversized agglomerates is at the most 20% w/w such as, e.g., at the most about 18% w/w such as, e.g., at the most about 15% w/w, at the most about 13% w/w, at the most about 10% w/w, at the most about 9%, w/w at the most about 8% w/w, at the most about 7% w/W, at the most about 6% w/w, at the most about 5% w/w, at the most about 4% w/w, at the most about 3% w/w or at the most about 2% wow based on the total weight of the coated particulate material.

The above-mentioned humidity range may suitably be determined by subjecting samples of the uncoated particulate material to a test, which involves coating the particulate material under conditions that involve changing the humidity in the coating chamber by changing the humidity of the inlet air or the amount of water in the coating liquid and determining the percentage of oversized particulate material for each humidity level. As mentioned above, this test is supplemented with the color test to ensure that the particles have a suitable mobility during the process so that e.g. occasional adherence to the equipment can be avoided (such occasional adherence may result in a coated product that is acceptable with respect to a low content of agglomerates, but which—from e.g. a manufacturing point of view—is unacceptable and may lead to products with poor/low reproducibility e.g. varying dissolution profiles).

To this end, one of the advantages of a method according to the present invention is that it is possible to obtain coated particulate material that has a particle size distribution that substantially equals that of the uncoated or precoated particulate material apart from a parallel displacement

Another advantages is that it is possible to obtain a coated particulate material (containing an active substance) that—when subject to an in vitro dissolution test—from batch to batch only has a small variation in dissolution of the active substance contained in the material.

Accordingly, a variation of at the most 10% is obtainable by use of the present method and careful adjusting the humidity range in the coating chamber. The permitted variability in release at any given time period should not exceed a total numerical difference of t 10% (in the following denoted % point) such as, e.g., at the most about ±7.5% or at the most about ±5% of the labeled content of the active substance (see CPMP (Commitee for proprietary medicinal products (EU) Guideline made by EMEA (The European Agency for the Evaluation of Medicinal Products): “Note for Guidance on quality of modified release products: A: oral dosage forms. B: transdermal dosage forms, section I (quality)”, CPMP/QWP/604/96, 29 Jul. 1999). The 10% point leads e.g. to a total variability of 20%: a requirement of 50+/−10% thus means an acceptance range from 40-60%. Normally, coating with organic solvents lead to coated products that have a higher variation.

The intra-batch variation with respect to dissolution rate is normally also very low such as, at the most about +10% RSD (relative standard deviation), at the most about ±7.5% RSD or at the most about +5% RSD. The determination is suitably performed by determining the time at which 50% of the total amount of active substance is released, and this time may then vary as described above with respect to relative standard variation (RSD).

In order to investigate whether a specific coated material has been subject to a method according to the invention, it may be possible to determine which ingredients that have been employed in the coating and to trace any residue of organic solvent e.g. by means of GC. If solvents like alcohols have been employed, open coating equipment has most likely been employed (the present method is suitable for such open systems). Furthermore, as has been described in details above, use of a method according to the invention leads to coated material that has a very high reproducibility/low variation in dissolution properties. However, other methods may also be applicable.

Particulate Material

The particulate material for use in a method of the invention may be an inert core or a core containing the active substance. It may also be in the form of beads, pellets, flake/flat pieces, granules, granulates, spheres or a tablet etc. It may also be in the form of crystals and may have any shape as mentioned above. The material that is coated may be water-insoluble or water-soluble; as mentioned above the method of the present invention is especially suitable in situation where at least part of the material to be coated is water-sensitive, but the method is also generally convenient

Examples of particulate material suitable for use according to the invention are, e.g., calcium alginate beads, cellulose spheres, charged resin spheres, glass beads, polystyrene spheres, sand silica beads or units, sodium hydroxide beads, sucrose spheres, collagen-based beads or flakes and crystals of an active substance.

In specific embodiments the particulate material is a core selected from cellulose spheres (an example of a water-insoluble material) and sucrose spheres (an example of a water-soluble material) pellets with L-HPC (an example of a water sensitive material).

The cellulose spheres may be obtained from:

Asahi Kasei Corporation

IPC Process Center eller Syntapharm

NP Pharm

The sucrose spheres may be obtained from:

Hanns G. Werner

Penwest

NP Pharm

In another specific embodiment, the particulate material is based on collagen and it may be in the form of a collagen-based core. The collagen-based bead or flakes is generally made of material derived from animals such as, e.g., horses, pigs, cows, etc., or from synthetic or semi-synthetic material. A suitable material for use is e.g. the collagen material disclosed in and prepared according to WO 02(070594 (Nycomed Pharma AS; entitled: “A method of preparing a collagen sponge, a device for extracting a part of a collagen foam, and an elongated collagen sponges”). The collagen material may be transformed into beads or flakes by means of lyophilizatlon or spray drying or any other appropriated method. The collagen core may have a form of a core, a sponge or foam, and it may be non-porous or porous. In the latter case it is suitable for inclusion of e.g. an active substance within the material.

As mentioned above, different types and/or different sizes of beads or flakes may behave differently in a coating process, which may be related to the differences in physico chemical properties (hydrophilicity, water content etc.). Accordingly, the coating conditions may be adjusted to each specific core material employed. In the examples herein specific conditions are given leading to suitable results. Furthermore, the specific coating appartus may have impact on the coating of the particulate material and the coating conditions should also be adjusted to the specific coating apparatus employed.

In general, a person skilled in the art can find guidance and advice or how to formulate and perform individual process step in Remington's Pharmaceutical Handbook to which reference is made.

The inventors have found that especially particulate material having a relatively low density and/or particle size leads to adherence and/or agglomeration problems.

In specific embodiments, the uncoated particulate material contains at the most about 15% w/w of water such as at the most about 10% w/w such as, e.g., at the most about 7.5% w/w, at the most about 7% w/v, at the most about 6% w/w, at the most about 5.5% w/w such as about 5% w/w. Normally, it is almost impossible to have a material that does not contain a certain small amount of water, and, as seen from the examples herein, the present method does not require that precautions are taken with respect to the water content of the starting materials. Thus, a small amount of water present in the starting material does not seem to affect the coating process. Furthermore, the coating composition in itself may also contain a certain concentration of water as long as this amount is taken into account when adjusting the relative humidity (RH) to the specific range within the coating chamber.

An example of a suitable particulate material for use in a method of the invention is e.g. cellulose spheres. In a specific embodiment, such cellulose spheres have a density of about 1.5 g/cm³.

Alternatively, the content of water in the particulate material is at the most about 5% w/w such as, e.g., at the most about 4.5% w/w, at the most about 4% w/w, at the most about 3.5% w/w, at the most about 3% w/w, at the most about 2.5% w/w such as about 2% w/w or 1% w/w. A suitable example is e.g. sucrose spheres.

During the coating process the water content of the particulate material may be reduced during the coating process. This is for example observed when cellulose spheres are coated with a method of the invention. The reduction in water content may be at least about 25% w/w such as, e.g., at least about 30% w/w, at least about 40% w/w, at least about 50% w/w, at least about 60% w/w, at least about 70% w/W or at least about 75% w/w.

In a specific embodiment, the particulate material may be essentially water insoluble such as, e.g., cellulose spheres, or it may essentially water soluble such as, e.g., sucrose spheres.

Solvents

The coating composition used in a method of the invention is based on an organic solvent selected from the group consisting of acetone, chloroform, dichoromethane, ethanol, ether, hexane, isopropanol, methanol, methyl acetate, methyl isobutyl ketone, methylene chloride, n-butanol, n-propanol, toluene, water, xylen, and mixtures thereof.

A method according to the invention is especially suitable for so called open coating systems and to this end especially alchols like e.g. methanol, ethanol, n-propanol, isopropanol, butanol, isobutanol, tert. butanol etc. or mixtures thereof are suitable as organic solvents.

In general ethanol, isopropanol and the like are preferred (if the process conditions makes it possible) as they are normally regarded as less harmless organic solvents.

Normally, the coating composition comprises at least about 70% v/v such as, e.g., at least about 75% v/v, at least about 80% v/v, at least about 85% v/v, at least about 90% v/v, at least about 95% v/v, at least about 97% v/v, at least about 99% v/v such as about 100% v/v of an organic solvent

The solvent of the coating composition may in certain cases contain up to about 30% v/v water or aqueous media. Normally water is not present in the solvent or only in concentrations below 25% v/v such as, e.g., at the most about 20% v/v, at the most about 15% v/v, at the most about 10% v/v, at the most about 5% or at the most about 2.5% v/v. As mentioned above, the content of water in the coating composition is then taken into account when adjusting the content of water in the inlet air in order to ensure that a correct humidity is obtained in the coating chamber.

Coating Conditions etc.

As mentioned above, a very critical parameter is the relative humidity. The relative humidity in the coating chamber during coating is at least about 20% such as, e.g., at least about 25% or at least about 30%.

The minimum relative humidity ensures that the static electricity within the coating chamber is kept at a level (if it is present at all) that does not impart any potential explosion risk and at the same time it ensures that the adherence of the particulate material to the coating equipment is of no significant importance with respect to the product properties obtained. Thus, the product obtained has high reproducibility and low variability with respect to e.g. dissolution characteristics. As mentioned herein before, the minimum relative humidity can be determined by use of the color test described herein and thus, it can be found for any suitable setting of the apparatus and coating condition.

The humidity of the chamber is controlled by adjusting the content of water in the inlet air and/or the water in the coating liquid that is delivered to the coating chamber. Thus, in those situations where the humidity of the inlet air is to low, the water content is adjusted by proper addition of water (this may e.g. be the situation during the winter season), and in those situations where the content of water is too high, the inlet air is dehumidified to a proper content of water before delivery to the coating chamber. A person skilled in the art will know how to adjust the water content in the air and in the coating composition in order to achieve the target value (or range) of relative humidity. Thus, taken the temperature of the air in the coating chamber into account, the content of water in the air is controlled.

As it appears from the Examples herein, the general guidance with respect to minimum relative humidity in the coating chamber seems to be applicable for all types of film-forming polymers. However, with respect to the upper limit of relative humidity it seems that this value is dependent on the specific apparatus, the film-forming polymer and the size of the particulate material etc. The upper value is of importance in order to avoid unwanted formation of agglomerates by overwetting and/or increased stickiness of the polymer film. A suitable test to determine the upper limit of the relative humidity in the coating chamber is described herein under the heading “Methods”.

However, from the examples described herein it seems that hydrophilic film-forming polymers that are suitable for use in an organic solvent based coating composition are less sensitive to the relative humidity provided that the relative humidity exceeds the minimum value, It may be contemplated that a relatively large relative humidity negatively can influence e.g. the inter- and/or intra-batch reproducibility and variability, and therefore, there may be situations where additional requirements with respect to these parameters are appropriate. In such situations, guidance is given herein in that respect. Generally, however, it seems that use of such hydrophilic film-forming polymers leads to suitable results if the relative humidity is in a range of from about 20 to about 100% relative humidity such as, e.g. in a range of from about 20 to about 95%, from about 20 to about 90%, from about 20 to about 85%, from about 25 to about 80%, from about 25 to about 75% or from about 30 to about 70%. Examples of hydrophilic film-forming polymers are e.g. those having a low standard viscosity such as, e.g., for ethylcellulose at the most 15 cps (e.g. ethylcellulose 7 cps etc. and HPMC-P). HPMC-P is a film-forminig polymer that requires a small amount of water present in the coating composition such as, e.g., at least about 5% w/w.

Film-forming polymers having medium/high standard viscosities (e.g. for ethylcelulose a standard viscosity of at least 15 cps) seem to require a relative humidity of at least 20%. A suitable range seems to be a range of from about 20 to about 60% such as. e.g. from about 20 to about 55%, from about 25 to about 55%, from about 30 to about 50%, from about 30 to about 45%, from about 30 to about 40%, from about 20 to about 30% or from about 25 to about 30%.

Normally, a suitable relative humidity should be determined with a view to the product temperature. With respect to the mentioned relative humidity, the temperature of the particulate material during coating is normally kept at a temperature in a range from about to about 60° C. such as, e.g., about 20 to about 50° C., from about 20 to about 45° C., from about 20 to about 40° C. or from about 20 to about 35° C.

In order to obtain a suitable relative humidity in the coating chamber the inlet air is adjusted to predetermined water content taken into consideration the temperature of the particulate material in the coating chamber.

In general, the water content of the inlet air is expressed by a dew point or gram water per kilo dry air.

In a specific embodiment, the temperature of the particulate material during coating is from about 26 to about 32° C., the dew point of the inlet air is from about 12 to about 14° C. and the relative humidity of the coating chamber is from about 28% to about 47%.

In another embodiment, the temperature of the particulate material during coating is from about 26 to about 32° C., the dew point of the inlet air is from about 14 to about 17 DC and the relative humidity of the coating chamber is from about 34% to about 50%.

In a further embodiment, the temperature of the particulate material during coating is from about 26 to about 32° C., the dew point of the inlet air is from about 7 to about 12° C. and the relative humidity of the coating chamber is from about 23% to about 35%.

Coating Compositions

The coating composition comprises a polymer such as a film-forming polymer. The coating may be a modified release coating, an immediate release coating, a taste-masking coating, an enteric coating, a coating containing an active substance such as, e.g., a poorly water-soluble or water-insoluble substance etc.

Suitably a film coating normally comprises a polymer selected from the group consisting of:

Ammonio methacryiate copolymer (Eudragit R L, Eudragit R S), cellulose acetate, cellulose acetate butyrate, cellulose acetate propionate, cellulose butyrate, cellulose propionate, cellulose valerate, crospovidone, ethyl cellulose, hydroxypropylcellulose, hydroxypropyl methyl cellulose (HPMC), hydroxyethylcellulose, polyacrylate dispersion, polydiethylaminomethylstyrene, polymethylstyrene, polyvinyl acetate, polyvinyl formal, polyvinyl butyryl, wax, amylose acetate phthalate, cellulose acetate phthalate CAP, cellulose acetate succinate, cellulose acetate trimellitate CAT, carboxymethyl ethylcellulose, formalin treated gelatine, hydroxypropyl methylcellulose acetate succinate HPMCAS, hydroxypropyl methylcellulose acetate phthalate, hydroxypropyl methylcellulose phthalate HPMC-P, meth acrylic acid copolymer (Eudragit L), methacrylic acid copolymer (Eudragit S), methacrylic acid copolymer (Eudragit FS), polyvinyl acetate phthalate PVAP (sureteric), shellac, starch acetate phthalate, styrene-maleic acid copolymer, zein, and mixtures thereof.

The coating composition may further contain one or more additives such as, e.g., a plasticizer, an antiadherence agent (e.g. PEG, talc, aerosil etc.), a taste-masking agent (flavour, aroma, color etc.), an enhancer, a stabilizer, a surfactant etc. It may also contain one or more active substances.

As ft appears from the examples herein, specific embodiments of the invention includes in the coating composition a substance that has hydrophilic nature and thereby may have a capacity of adsorbing water. Thus, a polyethylene glycol may be employed in a coating composition suitable for use in the method of the invention. The positive impact of polyethylene glycol (PEG, Macrogol) on the problem of static electricity is believed to be based on both its properties as a lubricant/anti-adhesive agent and it hydrophilic nature.

When it is advantageous to incorporate a polyethylene glycol it is an advantage if it is in the solid at the temperature at which the film is applied. Examples of suitable polyethylene glycols include PEG 1000, 1450. (1500) 1540, 2000, 3000, 3350, 4000, 4600, 6000, 8000, 20000, 35000.

Examples of other ingredients than polyethylene glycol that can be used as additive to a coating composition are e.g. talc, colloidal silica dioxide (Aerosil), polyoxyethylene alkyd ether, suppository base (e.g. cacao butter), hydrogenated castor oil, wax, paraffin, glycerol monostearate etc.

Moreover, pharmaceutically acceptable excipients having only glidant properties like hydrogenated castor oil and paraffin might have some impact on as well the product circulation during the coating process. The addition of a glidant might lead to a more freely flowing process which, In coating equipment like Wurster (bottom spray), might be able to “wash down” some or all of the particulate material that has adhered to the wall of the coating equipment due to static electricity. However, in coating equipment like the Rotor (tangential spray) this impact would be less.

Other Aspects of the Invention

The invention also relates to a coated particulate material obtainable by a method according to the invention and to the use a method according to the invention in the preparation of an enteric film, modified release or time controlled coated pharmaceutical composition. Applying active substances e.g. water-sensitive or poorly/not water soluble substances on uncoated or precoated particulate material

A method according to the invention may be used in the preparation of a pH and time-controlled drug delivery system for oral use comprising e.g. one or more of a first type of units, the first type of units comprising a therapeutically, prophylactically and/or diagnostically active substance, and the first type of units having a layered structure of at least

i) an inner core

ii) a time-controlled layer surrounding the inner core,

iii) a film coating applied on the time-controlled layer, wherein the film coating is substantially water insoluble but permeable to an aqueous medium, and

iv) an outer layer of an enteric coating.

Generally, the method may be used to apply at least one of layer ii), iii) and iv).

Active Substances

A method according to the present invention is especially suitable for the preparation of compositions containing an active drug substance. In principle any active substance may be incorporated in a composition prepared according to the Invention and the active substance may be contained in the particulate material that is coated by a method according to the Invention, it may be incorporated in the coating, it may be applied on top of the coating applied by the method of the Invention or it may be present in admixture with a coated material.

The term “active drug substance” encompasses the active substance in any suitable form.

Thus, the active substance may be a therapeutically, prophylactically and/or diagnostically active substance (drug substance) or it may be for cosmeceutical, nutriceutical or cosmetic use. In a specific embodiment of the invention, the active substance is a drug substance. The drug substance may be present in the form of a pharmaceutically acceptable salt, complex or prodrug thereof, or, whenever relevant, it may be present in racemic or any of its enantiomeric forms. Furthermore, it may be present in solid, semi-solid or dissolved form such as, e.g. in the form of particulate material e.g. In the form of crystals or it may be present in any amorphous or polymorphous form. Furthermore it may be presented as micronised powder or in the form of a solid dispersion.

Examples of active substances for use in a drug delivery system according to the invention are generally any active substance that is therapeutically, prophylactically and/or diagnostically active.

Other Additives

A drug delivery system according to the invention may further comprise one or more pharmaceutically acceptable exciplents. The one or more pharmaceutically acceptable exciplents may be present in any layer of the unit or added to the unit or units e.g. In order to enable compression of the units into e.g. tablets or in order to facilitate the manufacturing process and filling of the delivery system into a suitable dosage form (e.g. capsules, sachets etc.).

Suitable pharmaceutically acceptable exciplents are selected from the group consisting of fillers, diluents, binders and sweeteners.

Specific Examples Include:

Agar, alginate e.g. sodium alginate, calcium bicarbonate, calcium carbonate, calcium hydrogen phosphate, calcium phosphate, calcium sulphate, carboxyalkylcellulose, cellulose, charged sodium polystyrene sulphonate resin, dextran, dextrates, dextrin, dibasic calcium phosphate (Emcompress), ethyl cellulose, gelatine, glucose, glyceryl palmitostearate, gummi arabicum, hydroxyethyl cellulose, hydroxypropyl cellulose, hydroxypropylmethylcellulose, magnesium carbonate, magnesium chloride, magnesium oxide, maltodextrin, methylcellulose, microcrystalline cellulose, modified starches, polyethylene glycol, polyethylene oxide, polysaccharides e.g. dextran, polyvinylpyrrolidone (PVP), polyvinylpyrrolidone/vinyl acetate copolymer, soy polysaccharide, sodium carbonate, sodium chloride, sodium phosphate, starch, dextrose, fructose, glycerin, glucose, isomalt, lactitol, lactose, maltitol, maltose, mannitol, aorbitol, sucrose, tagatose, trehalose, xylitol, alitame, aspartame, acesulfam potassium, cyclamic acid, cyclamate salt (e.g. calcium cyclamate, sodium cyclamate), neohesperidine dihydrochalcone, thaumatin, saccharin, saccharin salt (e.g. ammonium saccharin, calcium saccharin, potassium saccharin, sodium saccharin), sucralose and mixtures thereof.

One or more excipients may also be added in order to improve the stability, the taste, the storage time etc. of the composition (or the active substance(s) contained in the composition) or to improve the bioavailability of the active substance(s) including the dissolution rate, the absorption rate and the extent of absorption.

METHODS

Method for Quantification the Degree of Agglomeration

The following method is used to determine the degree of agglomeration, cf. Claim 1 herein.

A representative sample is drawn from the coated product.

The sample is divided into fractions by use of a standard sieve analysis equipment using sieves having a size difference of approx. 100 μm.

These fractions are inspected with the purpose of determining the amount of agglomerates within each fraction. Agglomerates are defined as lumps consisting of two or more units of the original particulate material sticking together. The inspection is to be started with the fraction containing the smallest particles and can be done either visually followed by quantification based on weighing or by microscopy optionally combined with image analysis followed by quantification based on calculation of the volume of the equivalent spheres of the particles/agglomerates. The optimal choice of method for quantification depends on whether the use of microscopy has been appropriate.

The first fraction that has a content of more that 90% of agglomerates is identified.

The smaller screen size used to identify this fraction is used for dividing the whole batch of coated particulate material into two groups: good material and agglomerates.

These two groups of material are weighed and the amount of agglomerates in % (w/w) of the total amount of material is calculated.

Method for Quantification the Impact of Static Electricity

The following method is used to determine the impact of static electricity. Due to static electricity part of the particulate material will adhere to the coating equipment wall or other parts of the coating equipment during a coating process. This test is used to determine the lower limit of RH that is possible to apply and still achieve an acceptable result. As discussed hereinbefore, there are situations when even though the amount of agglomerates is acceptable, the result of the coating process will not be acceptable. This occurs when the film that has been applied has not been evenly distributed on the particles. This will occur if parts of the particles are adhering to the wall of the equipment (or other parts of the equipment) during the application of the coating. Therefore, a quantification of the amount of particles not participation in the product circulation is necessary. The below described test will be helpful in this context:

An amount of coating liquid corresponding to the application of a dry film having a thickness of a few microns is to be applied

To the coating liquid is added a coloring agent

After having applied this amount of film the coating process is stopped

The non colored and not fully colored material is isolated and quantified by weight

The amount of isolated material in % (w/w) of the total amount of material is calculated

In general, an acceptable result is achieved if the amount of isolated material based on the total amount of material is at the most about 10% w/w such as, e.g., at the most about 7.5% w/w or at the most about 5% w/w.

Another consequence of having part of the particulate material out of circulation during a coating process might be that the product that is actually circulating will become too wet. This will primarily happen if the coating liquid flow rate is maximised according the full batch size. In this situation agglomeration might also occur (see the method for quantification of degree of agglomeration).

### EXAMPLES

### Example 1

Preparation of cores with a Midodrine Containing Layer

2 kg cellulose, spheres with a particle size between 350-500 μm or between 500-700 μm were coated with a midodrine containing coat and an outer coat in a Glatt GPCG 3 fluid-bed equipped with a rotary processor (tangential spray). The nozzle was placed in the lowest position. The distance from the wall to the nozzle point was 2.5 mm and the nozzle port size was 1.2 mm. The spray pressure was 2.5 bar and the disc speed was 500 rpm. The product differential pressure across the product and slit was approximately 1.5 kPa.

The composition of the midodrine coating liquid (18.6% dry matter) and outer coat (8% dry matter) are shown in table A and S.

TABLE A

| Ingredients | Amount (g) |
|---|---|
| Midodrine hydrochloride | 172.5 |
| Hydroxypropyl methylcellulose E5 | 63.9 |
| Talc | 42.6 |
| Purified water | 1221.0 |
| Total | 1500.0 |

TABLE B

| Ingredients | Amount (g) |
|---|---|
| Hydroxypropyl methylcellulose E5 | 40.0 |
| Talc | 40.0 |
| Purified water | 920.0 |
| Total | 1000.0 |

In the coating process 11.7% w/w midodrine coat and 1% w/w outer coat were applied. The amount of dry matter applied is calculated in percentage of the core weight.

The cellulose spheres were heated to 40° C. and throughout the coating process the product temperature was kept at approximately 36° C. by adjustment of the liquid flow rate in the interval from 10 to 20 g/min. The inlet air temperature and the process airflow were kept at approximately 50° C. and 100 m³/h, respectively. The inlet air was dehumidified to a water content of 1 gram of water per kg dry air. Thereafter the coated cellulose spheres were dried for 15 minutes. The inlet air temperature was 55° C. and the disc speed was 350 rpm.

After coating, the coated cellulose spheres were screened through a screen size that was approximately 50% larger than the Dv(90) of the original particle size distribution. Agglomerates: less than 1%. The yield of Midodrine was at least 98%.

### Example 2

Preparation of Cores with L-HPC Layer Using Suspension Coating

1 kg precoated cores from Example 1 with a particle size between 350-500 μm were coated with L-HPC and an outer coat in a Glaft GPCG 3 fluid-bed equipped with a rotary processor. The composition of the suspension coat (25% dry matter) and the outer coat (4.2% dry matter) are shown in table C and D.

TABLE C

| Ingredients | Amount (g) |
|---|---|
| L-HPC LH-31 | 4472 |
| Hydroxypropyl cellulose L-/fine | 903 |
| Ethanol 99.9% | 16125 |
| Total | 21500 |

TABLE D

| Ingredients | Amount (g) |
|---|---|
| Hydroxypropyl cellulose L-/fine | 63.0 |
| Ethanol 99.9% | 1437.0 |
| Total | 1500.0 |

The cores were coated as described in Example 1 with the exception that the inlet air was humidified to control the relative humidity. The cores were heated to 25° C. and throughout the coating process the product temperature was kept at approximately 15° C. by adjustment of the liquid flow rate in the interval from 40 to 65 g/min. The inlet air temperature and the process airflow were kept at approximately 25° C. and 100 m³/h, respectively. The coated cores were dried on trays for approximately 24 hours at 40° C. The dried cores were fractionated by screening through a lower screen of 425 mm and an upper screen of 1000 mm.

### Example 3

Organic Based Coat

Examples of compositions of organic based coats with 8.5% dry matter, 9.9% dry matter and 8.1% dry matter are shown in table E, F and G, respectively. For other coating compositions see Table 1-4.

TABLE E

| Ingredients | Amount (g) |
|---|---|
| Ethyl cellulose 20 | 281.5 |
| Colloidal silica dioxide (Aerosil) | 56.5 |
| Ethanol 99.9% | 3662.0 |
| Total | 4000.0 |

TABLE F

| Ingredients | Amount (g) |
|---|---|
| Ethyl cellulose 20 | 281.5 |
| Polyethylene glycol 6000 (Macrogol) | 61.7 |
| Colloidal silica dioxide (Aerosil) | 56.5 |
| Ethanol 99.9% | 3600.3 |
| Total | 4000.0 |

TABLE G

| Ingredients | Amount (g) |
|---|---|
| Hydroxypropyl methylcellulose phthalate | 240.0 |
| Triethyl citrate | 12.0 |
| Colloidal silica dioxide (Aerosil) | 72.0 |
| Purified water | 551.4 |
| Ethanol 99.9% | 3124.6 |
| Total | 4000.0 |

### Example 4

Preparation of Cores With an Organic Based Coat Applying a Rotary Processor

2 kg cores with a midodrine containing layer or L-HPC layer were coated as described in Example 1 with the exception that the inlet air was humidified to control the relative humidity (se Table 1-4). The cores were heated to 30° C. and throughout the coating process the product temperature was maintained substantially in the interval from 28 to 31° C. by adjustment of the liquid flow rate in the interval from 10 to 20 g/min (see Table 1-4).

The inlet air temperature and the process airflow were kept at approximately 35° C. and 100 m³/h, respectively. The coated cores were dried for 15 minutes. The coated cores were screened through a screen size that was approximately 50% larger than the Dv(90) of the original particle size distribution. The amount of agglomerates, see Table 1-4.

### Example 5

Preparation of Cores With an Organic Based Coat Applying a Wurster

2 kg cores with a midodrine containing layer or L-H PC layer were coated a Glatt GPCG 3 fluid-bed equipped with a Wurster (bottom spray). A bottom plate Glatt type B and a gab size of 15 mm were applied. The nozzle port size was 1.2 mm and the spray pressure was 2.0 bars.

The inlet air was humidified to control the relative humidity (see Table 1-4). The cores were heated to 30° C. and throughout the coating process the product temperature was maintained substantially in the interval from 28 to 31° C. by adjustment of the liquid flow rate in the interval from 10 to 20 g/min (see Table 1-4). The inlet air temperature and the process airflow were kept at approximately 35° C. and 100 m³/h, respectively. The coated cores were dried for 15 minutes. The coated cores were screened through a screen size that was approximately 50% larger than the Dv(90) of the original particle size distribution.

The amount of agglomerates, see Table 1-4.

### Example 6

Dissolution Test

The in vitro release of midodrine was determined for batch 23100331 and batch 24100331 applying an in vitro dissolution method according to USP or Ph.Eur. The in vitro dissolution test method was:

| | |
|---|---|
| Dissolution medium: | 0.1N Hydrochloride |
| Media Temperature: | 37° C. ± 0.5° C. |
| Agitation: | 100 rpm |
| Detection system: | UV |

The result Is calculated by the use of a reference standard of midodrine. The result is reported as the average of three determinations (see FIG. 1). From the Figure it is seen that the batch prepared at a RH of 30% in the coating chamber differs from that prepared at a RH of 60% in the coating chamber. Thus, although the content of agglomerates in the RH 30% batch Is acceptable it seems as if a more pronounced retardation can be obtained when the coating is operating at a larger RH.

The circulation of the material at RH 30% does not seem to have been satisfactory, whereas this was the case for RH 60%. This difference was already observed dunng the coating, but was originally considered as being of no importance.

TABLE 1
Experiment with Ethyl Cellulose 7 cps

| | | Coat Composition | | Particulate | Relative | Liquid Flow | Product | Agglomerates | Coating |
|---|---|---|---|---|---|---|---|---|---|
| No. | Batch No. | Polymer (%)¹ | Excipients (%)² | Material | Humidity (%) | (g/min) | Temperature (° C.) | (%) | Process |
| 1 | 09080231 | 7 | Aerosil 20% | Pellets | 42-46 | 14.5-19 | 27-29 | <1 | Tangential |
| | | | Macrogol 21.9% | 500-800 μm | | | | | Spray |
| 2 | 22080231 | 7 | Aerosil 20% | Pellets | 43-48 | 15-19.5 | 24-28 | <1 | Tangential |
| | | | Macrogol 21.9% | 600-800 μm | | | | | Spray |
| 3 | 03090231 | 7 | Aerosil 20% | Pellets | 35-46 | 15.5-19 | 25-29 | 6.3 | Tangential |
| | | | | 500-800 μm | | | | | Spray |
| 4 | 14100331 | 7 | Aerosil 20% | 350 | 43 | 16.5-19 | 27-29 | <1 | Tangential |
| | | | Macrogol 21.9% | | | | | | Spray |
| 5 | 14100332 | 7 | Aerosil 20% | 350 | 50 | 16-19 | 28-29 | <1 | Tangential |
| | | | Macrogol 21.9% | | | | | | Spray |
| 6 | 15100331 | 7 | Aerosil 20% | 350 | 60 | 16.5-19 | 28-30 | <1 | Tangential |
| | | | Macrogol 21.9% | | | | | | Spray |
| 7 | 15100332 | 7 | Aerosil 20% | 350 | 30 | 16.5-19.5 | 28-29 | <1 | Tangential |
| | | | Macrogol 21.9% | | | | | | Spray |
| 8 | 15100333 | 7 | Aerosil 20% | 350 | 20 | 16.5-19.5 | 28-29 | * | Tangential |
| | | | Macrogol 21.9% | | | | | | Spray |
| 9 | 23100331 | 7 | Aerosil 20% | 350 | 30 | 15.5-19 | 27-31 | 2.9 | Tangential |
| | | | Macrogol 21.9% | | | | | | Spray |
| 10 | 24100331 | 7 | Aerosil 20% | 350 | 60 | 15-19.5 | 28-30 | 11.6 | Tangential |
| | | | Macrogol 21.9% | | | | | | Spray |
| 11 | 28100331 | 7 | Aerosil 20% | 350 | 60 | 14.5-18.5 | 28-29 | <1 | Tangential |
| | | | | | | | | | Spray |

¹% dry matter
²% dry matter of the polymer
Pellets = Cellets 350 coated with L-HPC
350 = Cellets 350-500 μm
*Static electricity about 1/4 of the particulate material were adhered to the coating equipment. The product circulation was not suitable for coating

TABLE 2
Experiment with Ethyl Cellulose 20 cps

| | | | | | | | Product | | |
|---|---|---|---|---|---|---|---|---|---|
| | | Coat Composition | | Particulate | Relative | Liquid Flow | Temperature | Agglomerates | Coating |
| No. | Batch No. | Polymer (%)¹ | Excipients (%)² | Material | Humidity (%) | (g/min) | (° C.) | (%) | Process |
| 1 | 10072002 | 3.5 | Aerosil 20% | 500 | 47-53 | 17 | 28-30 | 11 | Tangential |
| | | | Macrogol 20% | | | | | | Spray |
| 2 | 11072002 | 3.5 | Aerosil 20% | 500 | 27-29 | 16.5 | 30-31 | 2.8 | Tangential |
| | | | Macrogol 20% | | | | | | Spray |
| 3 | 12072002 | 5 | Aerosil 20% | 500 | 29-31 | 16 | 30-31 | 2.6 | Tangential |
| | | | Macrogol 20% | | | | | | Spray |
| 4 | 17072002 | 7 | Aerosil 20% | 500 | 30 | 18.5 | 30 | 5.5 | Tangential |
| | | | Macrogol 20% | | | | | | Spray |
| 5 | 27080231 | 7 | Aerosil 20% | Pellets | 41-51 | 14-18.5 | 25-29 | 12.6 | Tangential |
| | | | Macrogol 21.9% | 600-800 μm | | | | | Spray |
| 6 | 26090231 | 7 | Aerosil 20% | Pellets | 30-33 | 14.5-19 | 24.5-28.5 | 3 | Tangential |
| | | | Macrogol 21.9% | 0.6-1.0 mm | | | | | Spray |
| 7 | 02100231 | 7 | Aerosil 20% | Pellets | 35-48 | 13-19 | 24-28 | 33 | Tangential |
| | | | Macrogol 5% | 0.6-1.0 mm | | | | | Spray |
| 8 | 16100231 | 7 | Aerosil 20% | Pellets | 37.5-40 | 14-16.5 | 28-29 | 11.6 | Tangential |
| | | | Macrogol 35% | 0.6-1.0 mm | | | | | Spray |
| 9 | 10100331 | 7 | Aerosil 20% | 350 | 30 | 15-18 | 29-30 | 4.2 | Tangential |
| | | | Macrogol 21.9% | | | | | | Spray |
| 10 | 13100331 | 7 | Aerosil 20% | 350 | 20 | 16-18 | 29 | <1* | Tangential |
| | | | Macrogol 21.9% | | | | | | Spray |
| 11 | 21100331 | 7 | Aerosil 20% | 350 | 20 | 21.5-25 | 26-30 | 53.7* | Tangential |
| | | | Macrogol 21.9% | | | | | | Spray |

¹% dry matter
²% dry matter of the polymer
500 = Cellets 500-700 μm
Pellets = Cellets 350 coated with L-HPC
350 = Cellets 350-500 μm
*Static electricity about 1/3 of particulate material was adhered to the coating equipment. The product circulation was not suitable for coating

TABLE 3
Experiment with Ethyl Cellulose 7 & 20 cps (1:1)

| | | Coat Composition | | Particulate | Relative | Liquid Flow | Product | Agglomerates | Coating |
|---|---|---|---|---|---|---|---|---|---|
| No. | Batch No. | Polymer (%)¹ | Excipients (%)² | Material | Humidity (%) | (g/min) | Temperature (° C.) | (%) | Process |
| 1 | 24100231 | 7 | Aerosil 20% | Pellets | 36-38 | 14.5-18 | 24.5-29 | 1.6 | Tangential |
| | | | Macrogol 35% | 0.6-1.0 mm | | | | | Spray |
| 2 | 27110231 | 7 | Aerosil 20% | Pellets | 37.5-39 | 13-20 | 27.5-29 | 3 | Tangential |
| | | | Macrogol 35% | 0.6-1.0 mm | | | | | Spray |
| 3 | 05120231 | 7 | Aerosil 20% | Pellets | 35-39.5 | 15-19.5 | 27-28 | 1.5 | Tangential |
| | | | Macrogol 35% | 0.6-1.0 mm | | | | | Spray |
| 4 | 17120231 | 7 | Aerosil 20% | Pellets | 36.5-38.5 | 16 | 27-28.5 | <1 | Tangential |
| | | | Macrogol 35% | 0.6-1.0 mm | | | | | Spray |

¹% dry matter
²% dry matter of the polymer
Pellets = Cellets 350 coated with L-HPC

TABLE 4
Experiment with HPMCP HP 50

| | | | | | | | Product | | |
|---|---|---|---|---|---|---|---|---|---|
| | | Coat Composition | | Particulate | Relative | Liquid Flow | Temperature | Agglomerates | Coating |
| No. | Batch No. | Polymer (%)¹ | Excipients (%)² | Material | Humidity (%) | (g/min) | (° C.) | (%) | Process |
| 1 | 220702 | 6³ | Aerosil 20% | 500 | 40-45 | 12.5-16 | 27-29 | 12 | Tangential |
| | | | | | | | | | Spray |
| 2 | 260702 | 6⁴ | Aerosil 30% | 500 | 36-42 | 15-20 | 25.5-28.5 | 5.9 | Tangential |
| | | | | | | | | | Spray |
| 3 | 290702 | 6⁴ | Talc 50% | 500 | 49-54 | 17 | 26-28 | 21 | Tangential |
| | | | | | | | | | Spray |
| 4 | 310702 | 6⁴ | Aerosil 30% | 500 | 51-58 | 17 | 27-30 | 11.4 | Tangential |
| | | | | | | | | | Spray |
| 5 | 010802 | 6⁴ | Aerosil 30% | 500 | 52.5-62.5 | 17 | 26-30 | 19.3 | Tangential |
| | | | | | | | | | Spray |
| 6 | 050902 | 6⁴ | Aerosil 30% | 500 | 40.5-50 | 16-18 | 25-29 | 12.2 | Tangential |
| | | | | | | | | | Spray |
| 7 | 060902 | 6⁴ | Aerosil 30% | 500 | 53-63 | 15.5-18 | 25-27 | 37 | Tangential |
| | | | | | | | | | Spray |
| 8 | 05110231 | 6⁴ | Aerosil 30% | Pellets | 53-56 | 16.5-19 | 25-26.5 | 5.4 | Bottom Spray |
| | | | | 0.6-1.0 mm | | | | | |
| 9 | 12120231 | 6⁴ | Aerosil 30% | Pellets | 53-59 | 15-17 | 23-25.5 | 2.5 | Bottom Spray |
| | | | | 0.6-1.0 mm | | | | | |
| 10 | 23040331 | 6⁴ | Aerosil 30% | Pellets | 54.5-58.5 | 14-19 | 27-33 | 3.2 | Bottom Spray |
| | | | | 0.6-1.0 mm | | | | | |
| 11 | 10070331 | 6⁴ | Aerosil 30% | Pellets | 52-58 | 19 | 24-25.5 | <1 | Bottom Spray |
| | | | | 0.6-1.0 mm | | | | | |
| 12 | 16100335 | 6⁴ | Aerosil 30% | 350 | 60 | 16-17.5 | 25-27 | <1 | Bottom Spray |
| 13 | 16100336 | 6⁴ | Aerosil 30% | 350 | 53 | 16-17 | 24.5-28.5 | 4.3 | Bottom Spray |
| 14 | 17100331 | 6⁴ | Aerosil 30% | 350 | 73 | 16.4 | 26-28 | <1 | Bottom Spray |
| 15 | 20100331 | 6⁴ | Aerosil 30% | 350 | 5 | 15.5-17 | 26 | <1* | Bottom Spray |
| 16 | 22100331 | 6⁴ | Aerosil 30% | Pellets | 5 | 16-17 | 25.5-27 | <1** | Bottom Spray |
| | | | | 0.6-1.0 mm | | | | | |

¹% dry matter
²% dry matter of the polymer
³30% water in the liquid coat
⁴15% water in the liquid coat
500 = Cellets 500-700 μm
Pellets = Cellets 350 coated with L-HPC
350 = Cellets 350-500 μm
*Very Static electric - no homogeneous coating
**Static electricity - during drying the particulate material was very static electric

From Tables 14 it is seen that if the relative humidity in the coating chamber is below 20%, then the coating is not satisfactory (i.e. static electricity or adherence to the coating equipment occur) With respect to a suitable upper limit for the relative humidity in the coating chamber, it depends on the specific type of film-forming polymer(s) employed. Thus, there are Indications that film-forming polymers having a low standard viscosity (for ethylcellulose, at the most 15 cps) do not have an upper limit, whereas film-forming polymers having a medium/high standard viscosity seem to have an upper limit of about 60% RH. In the paragraph headed “Methods”, two tests are described that enable determination of which relative humidity or range of relative humidity that should be used under a given set of operating conditions (coating equipment, type of film-forming polymer, type and size of particulate material etc.).

From Table 1 the following conclusions can be made:

When the RH is as low as 20% RH (batch No. 8) the static electricity is significant leading to non-optimal product circulation.

When looking at batch No. 3 is can be seen that the absence of macrogol may lead to an increased degree of agglomeration. However, when RH is increased, agglomeration is avoided even though macrogol Is not present.

From Table 2 the following conclusion can be made:

When the RH is as low as 20% RH (batch Nos. 10 and 11) the static electricity is significant, leading to non-optimal product circulation. When a significant part of the batch is out of circulation the risk of agglomeration is severe as can be seen from batch No. 11.

When the RH is increased to 30% RH (batch No. 9) the negative impact of the static electricity is low.

When the RH is increase further to close to 50% (batch Nos. 1 and 5) a more pronounced degree of agglomeration is seen. This is in contrast to what was seen at RH 60% for EC7 and illustrates the more hydrophobic nature and higher degree of stickiness of the EC20 polymer.

In Table 3 is given examples of application of a mixture (1:1) of EC7 and EC20.

In Table 4 the following conclusion can be made:

When looking at tangential spray the optimal RH is close to 30-40% RH (batch No. 2). When this RH is increased the agglomeration is increased too. (batch Nos. 4, 5, 6 7). If the Aerosil is substituted with talc further agglomeration takes place.

When the coating principle Is changed to bottom spray the risk of agglomeration is much less even though the mean particle size is smaller. For bottom spray based application no upper RH limit with respect to agglomeration is evident a When the RH is as low as 5% RH (batch No. 15), the static electricity is significant leading to non optimal product circulation

When RH is increased to 50% or more (batch Nos. 13, 12 and 14), the negative impact of the static electricity is low for bottom spray, but not for tangential spray (batch Nos. 4-7).

It is more easy to coat larger particle than smaller particle

1. A method for coating a particulate material for pharmaceutical, cosmeceutical, nutriceutical or cosmetic use or for use in food or food stuff, the coating being performed in a coating equipment, which comprises a coating chamber having
i) means for supply of a coating composition, and
ii) means for supply of inlet air to provide a flaw of inlet air,
the method comprises
i) loading uncoated or pre-coated particulate material into the coating chamber,
ii) providing a flow of inlet air that has been adjusted so that the humidity of the air in the coating chamber ensures that unwanted agglomeration of the particulate material and/or adherence to the coating equipment are substantially reduced or avoided during the coating process, and
iii) spraying on the particulate material a coating composition comprising a solvent that contains at least about 70% v/v of one or more organic solvents and at the most about 30% v/v of an aqueous medium, to obtain coated particulate material containing at the most about 20% w/W agglomerates.

2. A method according to claim 1, wherein the weight fraction of oversized agglomerates is at the most about 18% wow such as, e.g., at the most about 15% w/w, at the most about 13% w/w, at the most about 10% w/w, at the most about 9% w/w, at the most about 8% w/w, at the most about 7% w/w, at the most about 6% w/w, at the most about 5% w/w, at the most about 4% w/w, at the most about 3% w/w or at the most about 2% w/w based on the total weight of the coated particulate material.

3. A method according to claim 1 or 2, wherein the relative humidity in the coating chamber is adjusted to a value determined by subjecting samples of the uncoated or pre-coated particulate material to a test involving coating the particulate material under conditions involving changing the humidity of the air in the coating chamber and determining for each humidity level the percentage of oversized particulate material.

4. A method according to any of the preceding claims, wherein the relative humidity in the coating chamber is adjusted to a value that is equal to or larger than a minimum value so that adhesion of the particulate material to the coating chamber is substantially avoided.

5. A method according to claim 4, wherein the minimum value of the relative humidity in the coating chamber is determined by subjecting the uncoated or pre-coated particulate material to a color test as described herein and the minimum value being expressed as the relative humidity at which at the most about 10% w/w of the particulate material subjected to the color test is not colored or not fully colored.

6. A method according to claim 5, wherein the minimum value of the relative humidity is expressed as the relative humidity at which at the most about 7.5% w/w or at the most about 5% w/w of the particulate material is not colored or not fully colored.

7. A method according to any of the preceding claims, wherein the relative humidity in the coating chamber is at least about 20% such as, e g., at least about 25% or at least about 30%.

8. A method according to any of the preceding claims, wherein the density of the uncoated or precoated particulate material is at the most about 3.0 g/cm³, the density being determined by a mercury intrusion method.

9. A method according to claim 8, wherein the density of the uncoated or precoated particulate material is at the most about 2.0 g/cm³ such as, e.g., at the most about 1.9 g/cm³, at the most about 1.8 g/cm³, at the most about 1.7 g/cm³, at the most about 1.6 g/cm³, at the most about 1.55 g/cm³, at the most about 1.5 g/cm³ or at the most about 1.4 g/cm³.

10. A method according to any of the preceding claims, wherein the mean particle size of the uncoated or precoated particulate material is at the most about 1400 μm.

11. A method according to claim 10, wherein the mean particle size of the uncoated or precoateel particulate material is at the most about 1200 μm such as, e.g., at the most about 1100 μm, at the most about 1000 μm, at the most about 900 μm, at the most about 800 μm, at the most about 750 μm, at the most about 700 μm, at the most about 650 μm, at the most about 600 μm, at the most 550 μm or at the most about 500 μm; such as, e.g., from about 150 μm to about 1200 μn, from about 200 μm to about 1200 μm from about 2000 μm to about 1000 μm, from about 250 μm to about 800 μm or from about 300 μm to about 750 μm.

12. A method according to any of the preceding claims, wherein the uncoated or precoated particulate material contains at the most about 15% w/w of water.

13. A method according to claim 12, wherein the content of water is at the most about 10% w/w such as, e.g., at the most about 7.5% w/w, at the most about 7% w/w, at the most about 6% w/w, at the most about 5.5% w/w such as about 5% w/w.

14. A method according to claim 12, wherein the content of water Is at the most about 5% w/w such as, e.g., at the most about 4.5% w/w, at the most about 4% w/w, at the most about 3.5% w/w, at the most about 3% w/w, at the most about 2.5% w/w such as about 2% w/w or about 1% w/w.

15. A method according to any of the preceding claims, wherein the particulate material is selected from pharmaceutically, cosmeceutically, nutriceutically and/or cosmetically acceptable beads, spheres, granules, granulates, flakes and pellets.

16. A method according to any of the preceding claims, wherein the particulate material Is in the form of a core that is selected from calcium alginate beads, cellulose spheres, charged resin spheres, glass beads, polystyrene spheres, sand silica beads or units, sodium hydroxide beads, sucrose spheres, collagen-based beads, and collagen-based flakes.

17. A method according to any of claims 1-14, wherein the particulate material is made of crystals of an active substance.

18. A method according to claim 16, wherein the cores are selected from cellulose spheres and sucrose spheres.

19. A method according to claim 16, wherein the cores are collagen-based beads or flakes.

20. A system according to claim 16 or 19, wherein the collagen-based beads or flakes are made of material derived from animals such as, e.g., horses, pigs, cows, etc., or from synthetic or semi-synthetic material.

21 A method according to any of the preceding claims, wherein the particulate material is essentially water insoluble.

22. A method according to claim 21, wherein the particulate material is cellulose spheres.

23. A method according to any of claims 1-20, wherein the particulate material is essentially water soluble.

24. A method according to claim 23, wherein the particulate material is sucrose spheres.

25. A method according to any of the preceding claims, wherein the coating composition comprises an organic solvent selected from the group consisting of: methanol, ethanol, n-propanol, isopropanol, n-butanol, isobutanol, tert. butanol and other alcohols. And mixtures thereof.

26. A method according to any of the preceding claims, wherein the coating composition comprises at least about 80% v/v such as, e.g., at least about 85% v/v, at least about 90% v/v, at least about 95% v/v, at least about 97% v/v, at least about 99% v/v such as about 100% v/v of an organic solvent.

27. A method according to any of the preceding claims, wherein temperature of the particulate material during coating is kept at a temperature in a range from about 20 to about 60° C.

28. A method according to any of the preceding claims, wherein temperature of the particulate material during coating is kept at a temperature in a range from about 20 to about 50° C. such as, e.g. from about 20 to about 45° C., from about 20 to about 40° C. or from about 20 to about 35° C.

29. A method according to any of the pricing claims, wherein the relative humidity In the coating chamber during coating is from about 20% to about 100%.

30. A method according to any of the preceding claims, wherein the coating composition comprises a film-forming polymer that has a low standard viscosity and the relative humidity in the coating chamber is in a range of from about 20 to about 95%, from about 20 to about 90%, from about 20 to about 85%, from about 25 to about 80%. From about 25 to about 75% or from about 30 to about 70%.

31. A method according to any of claims 1-28, wherein the coating composition comprises a film-forming polymer that has a medium/high standard viscosity and the relative humidity in the coating chamber is from about from about 20 to about 60% such as, e.g., from about 20 to about 55%, from about 25 to about 65%, from about 30 to about 50%, from about 30 to about 45%. From about 30 to about 40%, from about 20 to about 30% or from about 20 to about 25%.

32. A method according to any of the preceding claims, wherein the coating composition comprises an additive such as, e.g., an antiadherent agent.

33. A method according to any of the preceding claims, wherein the coating composition comprises a pharmaceutically acceptable excipient that has a hydrophilic nature.

34. A method according to any of the preceding claims, wherein the coating composition comprises a polyethylene glycol.

35. A method according to claim 34, wherein the polyethylene glycol Is solid at room temperature.

36. A method according to any of the preceding claims, wherein the coating composition comprises at least about 5% w/w of a polyethylene glycol.

37. A method according to any of claims 1-18, 21, 25-36, wherein the particulate material is cellulose spheres having a density of about 1.5 g/cm³.

38. A method according to any of claims 1-18, 23-36, wherein the particulate material is sucrose spheres.

39. A coated particulate material obtainable by a method according to any of claims 1-36.

40. Use of a method according to any of claims 1-38 in the preparation of an enteric coated pharmaceutical composition.

41. Use of a method according to any of claims 1-38 in the preparation of a film coated pharmaceutical composition.

42. Use of a m thod according to any of claims 1-38 In the preparation of a modified release pharmaceutical composition.

43. Use of a method according to any of claims 1-38 in the preparation of a time-controlled pharmaceutical composition.

44. Use of a method according to any of claims 1-38 in the preparation of a pharmaceutical composition comprising an ingredient that is water-sensitive and/or poorly water-soluble or water-insoluble.

45. Use according to claim 44, wherein the ingredient is an active substance.
